(19) United States
(12) Patent Application Publication
(10) Pub. No.: US 2023/0000542 A1
(43) Pub. Date: Jan. 5, 2023

(54) ELECTROSURGICAL TOOL WITH CAPACITIVE COUPLING MITIGATION SHEATH ASSEMBLY

(71) Applicant: Cilag GmbH International, Zug (CH)

(72) Inventor: Niko J. Murrell, Evendale, OH (US)

(21) Appl. No.: 17/363,578

(22) Filed: Jun. 30, 2021

(51) Int. Cl. A61B 18/14

(52) U.S. Cl. CPC A61B 18/1445; A61B 2018/1253

(57) ABSTRACT

A monopolar surgical tool is disclosed. The monopolar surgical tool comprises a shaft assembly comprising an electrically-conductive proximal portion and an electrically-insulative distal portion comprising a distal end. The monopolar surgical tool further comprises an end effector coupled to the distal end, an RF conductor extending distally through the shaft assembly to the end effector, and a guard electrode extending distally through the shaft assembly to an electrical contact region along the electrically-insulative distal portion, and a multi-layer sheath assembly. The multi-layer sheath assembly comprises an electrically-insulative inner layer surrounding a portion of the end effector and extending proximally along a portion of the shaft assembly, an electrically-insulative outer layer, and an electrically-conductive layer positioned between the electrically-insulative inner layer and the electrically-insulative outer layer. The electrically-conductive layer provides a return path for leaked current to the electrical contact region and along the guard electrode within the shaft assembly.

### BACKGROUND

The present disclosure relates to surgical systems, surgical devices, and surgical techniques. Surgical devices include electrosurgical devices, such as robotic surgical tools and handheld surgical instruments, for example, which can include an energizable end effector and/or component thereof.

### SUMMARY

In one general aspect, the present disclosure provides a monopolar surgical tool, comprising a shaft assembly comprising an electrically-conductive proximal portion and an electrically-insulative distal portion comprising a distal end. The monopolar surgical tool further comprises an end effector coupled to the distal end, an RF conductor extending distally through the shaft assembly to the end effector, and a guard electrode extending distally through the shaft assembly to an electrical contact region along the electrically-insulative distal portion, wherein the guard electrode surrounds the RF conductor along a length thereof. The monopolar surgical tool further comprises a multi-layer sheath assembly. The multi-layer sheath assembly comprises an electrically-insulative inner layer surrounding a portion of the end effector and extending proximally along a portion of the shaft assembly, an electrically-insulative outer layer, and an electrically-conductive layer positioned between the electrically-insulative inner layer and the electrically-insulative outer layer. The electrically-conductive layer extends proximally to the electrical contact region. The electrically-conductive layer provides a return path for leaked current to the electrical contact region and along the guard electrode within the shaft assembly.

In another aspect, the present disclosure provides a monopolar surgical tool, comprising a shaft, an electrically-conductive end effector comprising an articulation joint and a distal end, wherein the distal end is configured to articulate relative to the shaft at the articulation joint, and an RF conductor extending distally through the shaft to the electrically-conductive end effector. The monopolar surgical tool further comprises a guard electrode extending distally through the shaft to an electrical contact region, wherein the guard electrode surrounds the RF conductor along a length thereof. The monopolar surgical tool further comprises a sheath assembly. The sheath assembly comprises an electrically-insulative outer sheath surrounding the articulation joint and extending proximally along a portion of the shaft, and an electrically-conductive inner sheath electrically isolated from the electrically-conductive end effector and encased by the electrically-insulative outer sheath. The electrically-conductive inner sheath extends proximally to the electrical contact region and is configured to provide a return path for leaked current from the electrically-conductive inner sheath along the guard electrode through the shaft.

In another aspect, the present disclosure provides a monopolar surgical tool for use with a robotic surgical system and a generator. The monopolar surgical tool comprises a shaft comprising an electrically-insulative distal portion, an electrically-conductive distal end effector extending distally from the shaft, and an electrical conductor extending through the shaft to the electrically-conductive distal end effector. The monopolar surgical tool further comprises a capacitive coupling mitigation system configured to provide a secondary return path to the generator. The capacitive coupling mitigation system comprises a conductive contact region defined through the electrically-insulative distal portion, and a conductive inner sheath extending proximally from the electrically-conductive distal end effector to the conductive contact region. The conductive inner sheath is electrically isolated from the electrically-conductive distal end effector. The capacitive coupling mitigation system further comprises a guard electrode extending proximally from the conductive contact region through the shaft to the generator.

### BRIEF DESCRIPTION OF THE FIGURES

The novel features of the various aspects are set forth with particularity in the appended claims. The described aspects, however, both as to organization and methods of operation, may be best understood by reference to the following description, taken in conjunction with the accompanying drawings in which:

FIG. 1 is a plan view of a surgical procedure depicting a cart-based robotic system arranged for diagnostic and/or therapeutic bronchoscopy procedure(s), in accordance with at least one aspect of the present disclosure.

FIG. 2 is a perspective view of a robotic arm cart of the cart-based robotic system of FIG. 1, in accordance with at least one aspect of the present disclosure.

FIG. 3 is a perspective view of a robotic arm having a tool driver and a paired robotic tool detached from the tool driver, in accordance with at least one aspect of the present disclosure.

FIG. 4 is another perspective view of the robotic arm of FIG. 3 having a tool driver and a paired robotic tool detached from the tool driver, in accordance with at least one aspect of the present disclosure.

FIG. 5 is a perspective view of a tool driver, in accordance with at least one aspect of the present disclosure.

FIG. 6 is an elevation view of a surgical tool for use with the tool driver of FIG. 5, in accordance with at least one aspect of the present disclosure.

FIG. 7 is an exploded perspective view of a distal portion of a robotic tool including an electrically-insulative outer sheath, in accordance with at least one aspect of the present disclosure.

FIG. 8A is an elevation view of the distal portion of the robotic tool of FIG. 7 depicting the electrically-insulative outer sheath in a first position, in accordance with at least one aspect of the present disclosure.

FIG. 8B is an elevation view of the distal portion of the robotic tool of FIG. 7 depicting the electrically-insulative outer sleeve in a second position, in accordance with at least one aspect of the present disclosure.

FIG. 9 is an elevation view of a distal portion of a robotic tool adjacent to patient tissue during a surgical procedure, in accordance with at least one aspect of the present disclosure.

FIG. 10 is an elevation, cross-section view of a distal portion of a robotic tool taken along a central plane, in accordance with at least one aspect of the present disclosure.

FIG. 11 is an elevation, cross-section view of the robotic tool of FIG. 10 taken along the plane indicated in FIG. 10, in accordance with at least one aspect of the present disclosure.

FIG. 12 is an elevation, cross-section view of the robotic tool of FIG. 10 taken along the plane indicated in FIG. 10, in accordance with at least one aspect of the present disclosure.

Corresponding reference characters indicate corresponding parts throughout the several views. The exemplifications set out herein illustrate various aspects of the present disclosure, in one form, and such exemplifications are not to be construed as limiting the scope of the invention in any manner.

### DETAILED DESCRIPTION

Applicant of the present application also owns the following U.S. Patent Applications, filed on even date herewith, each of which is herein incorporated by reference in its entirety:
U.S. Patent Application titled GRASPING WORK DETERMINATION AND INDICATIONS THEREOF, Attorney Docket No. END9325USNP1/210077;
U.S. Patent Application titled STAPLE CARTRIDGE REPLACEMENT, Attorney Docket No. END9327USNP1/210078; and
U.S. Patent Application titled LINK-DRIVEN ARTICULATION DEVICE FOR A SURGICAL DEVICE, Attorney Docket No. END9328USNP1/210079.

Applicant of the present application also owns the following U.S. Patent Applications, filed Dec. 30, 2020, each of which is herein incorporated by reference in its entirety:
U.S. patent application Ser. No. 17/137,829, titled SURGICAL TOOL WITH TOOL-BASED TRANSLATION AND LOCK FOR THE SAME;
U.S. patent application Ser. No. 17/137,846, titled ROBOTIC SURGICAL TOOLS HAVING DUAL ARTICULATION DRIVES;
U.S. patent application Ser. No. 17/137,852, titled TORQUE-BASED TRANSITION BETWEEN OPERATING GEARS; and
U.S. patent application Ser. No. 17/137,857, titled DUAL DRIVING PINION CROSSCHECK.

Applicant of the present application also owns U.S. patent application Ser. No. 16/587,744, filed Sep. 30, 2019, titled COMMUNICATING CLOSURE EFFORT FOR ROBOTIC SURGICAL TOOLS BACKGROUND, which published Apr. 1, 2021 as U.S. Patent Application Publication No. 2021/0093409, which is incorporated by reference herein in its entirety.

Applicant of the present application also owns U.S. patent application Ser. No. 16/553,725, filed Aug. 28, 2019, titled ARTICULATING INCLUDING ANTAGONISTIC CONTROLS FOR ARTICULATION AND CALIBRATION, which published Mar. 4, 2021 as U.S. Patent Application Publication No. 2021/0059777, which is incorporated by reference herein in its entirety.

Applicant of the present application also owns the following U.S. Patent Applications, filed on Mar. 29, 2018, each of which is herein incorporated by reference in its entirety:
U.S. patent application Ser. No. 15/940,627, titled DRIVE ARRANGEMENTS FOR ROBOT-ASSISTED SURGICAL PLATFORMS, which issued May 25, 2021 as U.S. Pat. No. 11,013,563;
U.S. patent application Ser. No. 15/940,676, titled AUTOMATIC TOOL ADJUSTMENTS FOR ROBOT-ASSISTED SURGICAL PLATFORMS, which published Jul. 4, 2019 as U.S. Patent Application Publication No. 2019/0201142; and
U.S. patent application Ser. No. 15/940,711, titled SENSING ARRANGEMENTS FOR ROBOT-ASSISTED SURGICAL PLATFORMS, which published Jul. 4, 2019 as U.S. Patent Application Publication No. 2019/0201120.

Applicant of the present application also owns U.S. Provisional Patent Application Ser. No. 62/611,339, titled ROBOT ASSISTED SURGICAL PLATFORM, filed Dec. 28, 2017, which is incorporated by reference herein in its entirety.

Application of the present application also owns U.S. patent application Ser. No. 13/118,241, titled SURGICAL STAPLING INSTRUMENTS WITH ROTATABLE STAPLE DEPLOYMENT ARRANGEMENTS, filed May 27, 2011, which issued Jul. 7, 2015 as U.S. Pat. No. 9,072,535, which is incorporated by reference herein in its entirety.

U.S. Pat. No. 10,470,830, titled SYSTEM AND METHODS FOR INSTRUMENT BASED INSERTION ARCHITECTURES, issued Nov. 12, 2019, is also incorporated by reference herein in its entirety.

Before explaining various aspects of a robotic surgical platforms and surgical devices in detail, it should be noted that the illustrative examples are not limited in application or use to the details of construction and arrangement of parts illustrated in the accompanying drawings and description. The illustrative examples may be implemented or incorporated in other aspects, variations, and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects, and/or examples.

Minimally-invasive surgery (MIS), such as laparoscopic surgery and bronchoscopy, typically involves techniques intended to reduce tissue damage during a surgical procedure. For example, laparoscopic procedures can involve creating a number of small incisions in the patient (e.g., in the abdomen) and introducing one or more surgical tools (e.g., end effectors and an endoscope) through the incisions into the patient. Bronchoscopy can involve passing a bronchoscope through a patient's nose and/or mouth, down the patient's throat, and into the patient's lungs. Surgical procedures may then be performed using the introduced surgical tools and with visualization aid provided by the endoscope, for example.

MIS may provide certain benefits, such as reduced patient scarring, less patient pain, shorter patient recovery periods, and/or lower medical treatment costs associated with patient recovery. Recent technological developments allow robotic systems to perform more MIS procedures. The robotic systems typically include one or more robotic arms for manipulating surgical tools based on commands from a remote operator (e.g. surgeon/clinician). A robotic arm may, for example, support at its distal end various surgical devices such as surgical end effectors, imaging devices, and cannulas for providing access to the patient's body cavity and organs.

Existing robotically-assisted surgical systems typically consist of a surgeon console and a patient-side cart with one or more interactive robotic arms controlled from the console. For example, one robotic arm can support a camera and the other robotic arm(s) can support robotic tools such as scalpels, scissors, graspers, and staplers, for example. Various exemplary robotic tools are further described herein.

A robotic surgical system disclosed herein can be a software-controlled, electro-mechanical system designed for clinicians to perform MIS procedures. The robotic surgical system can be used with an endoscope, compatible endoscopic instruments, and accessories. The system may be used by trained clinicians (e.g. physicians/surgeons) in an operating room environment to assist in the accurate control of compatible endoscopic instruments during robotically-assisted urologic, gynecologic, gastrological, and other laparoscopic surgical procedures. The compatible endoscopic instruments and accessories for use with the surgical system are intended for endoscopic manipulation of tissue including stapling, grasping, cutting, blunt and sharp dissection, approximation, ligation, electrocautery, and suturing, for example.

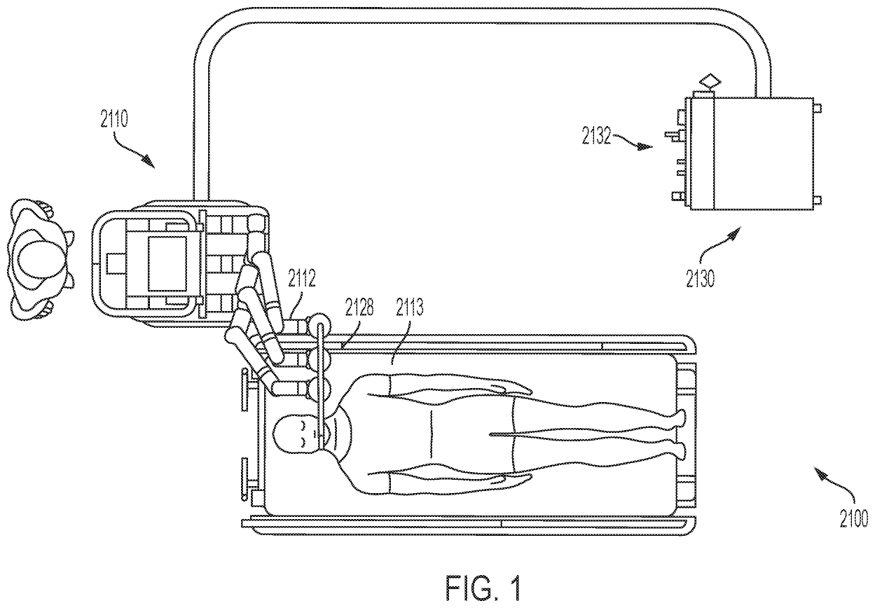

An exemplary robotic system 2100 is shown in FIG. 1, which depicts a cart-based robotically-enabled system arranged for a diagnostic and/or therapeutic bronchoscopy procedure. During a bronchoscopy, the system 2100 may include a cart 2110 having one or more robotic arms 2112 to deliver a surgical device, such as a steerable endoscope 2113, which may be a procedure-specific bronchoscope for bronchoscopy, to a natural orifice access point (i.e., the mouth of the patient positioned on a table in the present example) to deliver diagnostic and/or therapeutic tools. As shown, the cart 2110 may be positioned proximate to the patient's upper torso in order to provide access to the access point. Similarly, the robotic arms 2112 may be actuated to position the bronchoscope relative to the access point. The arrangement in FIG. 1 may also be utilized when performing a gastro-intestinal (GI) procedure with a gastroscope, a specialized endoscope for GI procedures.

With continued reference to FIG. 1, once the cart 2110 is properly positioned, the robotic arms 2112 may insert the steerable endoscope 2113 into the patient robotically, manually, or a combination thereof. The endoscope 2113 may be directed down the patient's trachea and lungs after insertion using precise commands from the robotic system until reaching the target destination or operative site. For example, the endoscope 2113 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 2113 may endoscopically deliver tools to resect the potentially cancerous tissue. In some instances, diagnostic and therapeutic treatments can be delivered in separate procedures. In those circumstances, the endoscope 2113 may also be used to deliver a fiducial to “mark” the location of the target nodule as well. In other instances, diagnostic and therapeutic treatments may be delivered during the same procedure.

The system 2100 may also include a movable tower 2130, which may be connected via support cables to the cart 2110 to provide support for controls, electronics, fluidics, optics, sensors, and/or power to the cart 2110. Placing such functionality in the tower 2130 allows for a smaller form factor cart 2110 that may be more easily adjusted and/or re-positioned by an operating clinician (e.g. surgeon) and his/her staff. Additionally, the division of functionality between the cart/table and the tower 2130 reduces operating room clutter and facilitates improving clinical workflow. While the cart 2110 may be positioned close to the patient, the tower 2130 may be stowed in a remote location to stay out of the way during a procedure.

In support of the robotic systems described above, the tower 2130 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 2130 or the cart 2110, may control the entire system or sub-system(s) thereof. For example, when executed by a processor of the computer system, the instructions may cause the components of the robotics system to actuate the relevant carriages and arm mounts, actuate the robotics arms, and control the robotic surgical tools. For example, in response to receiving the control signal, the motors in the joints of the robotics arms may position the arms into a certain posture.

The tower 2130 may also include a pump, flow meter, valve control, and/or fluid access in order to provide controlled irrigation and aspiration capabilities to the system that may be deployed through the endoscope 2113. These components may also be controlled using the computer system of tower 2130. In some aspects, irrigation and aspiration capabilities may be delivered directly to the endoscope 2113 through separate cable(s).

The tower 2130 may include a voltage and surge protector designed to provide filtered and protected electrical power to the cart 2110, thereby avoiding placement of a power transformer and other auxiliary power components in the cart 2110, resulting in a smaller, more moveable cart 2110.

The tower 2130 may also include support equipment for the sensors deployed throughout the robotic system 2100. For example, the tower 2130 may include opto-electronics equipment for detecting, receiving, and processing data received from the optical sensors or cameras throughout the robotic system 2100. In combination with the control system, such opto-electronics equipment may be used to generate real-time images for display in any number of consoles deployed throughout the system, including in the tower 2130. Similarly, the tower 2130 may also include an electronic subsystem for receiving and processing signals received from deployed electromagnetic (EM) sensors. The tower 2130 may also be used to house and position an EM field generator for detection by EM sensors in or on the robotic surgical tool. The tower 2130 can also house an electrosurgical generator for supplying RF current to a robotic surgical tool, such as monopolar scissors, for example.

The tower 2130 may also include a console 2132 in addition to other consoles available in the rest of the system, e.g., a console mounted on top of the cart 2110. The console 2132 may include a user interface and a display screen, such as a touchscreen, for the clinician. Consoles in the system 2100 are generally designed to provide both robotic controls as well as pre-operative and real-time information of the procedure, such as navigational and localization information of the endoscope 2113. When the console 2132 is not the only console available to the clinician, it may be used by a second clinician, such as a nurse, for example, to monitor the health or vitals of the patient and the operation of system, as well as provide procedure-specific data, such as navigational and localization information. In other aspects, the console 2132 is housed in a body that is separate from the tower 2130.

The tower 2130 may be coupled to the cart 2110 and endoscope 2113 through one or more cables or connections. In some aspects, the support functionality from the tower 2130 may be provided through a single cable to the cart 2110, simplifying and de-cluttering the operating room. In other aspects, specific functionality may be coupled in separate cabling and connections. For example, while power may be provided through a single power cable to the cart, the support for controls, optics, fluidics, and/or navigation may be provided through one or more separate cables.

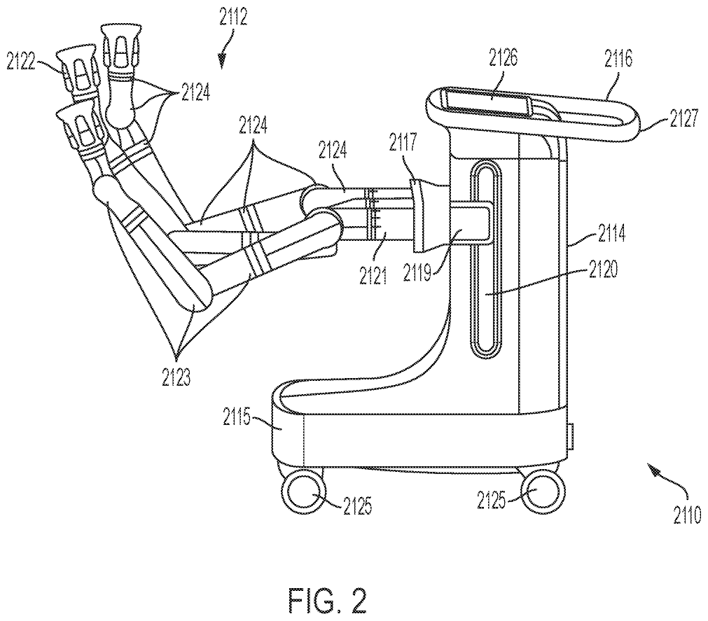

FIG. 2 depicts the cart 2110 from the cart-based robotically-enabled system 2100 shown in FIG. 1. The cart 2110 generally includes an elongated support structure 2114 (often referred to as a “column”), a cart base 2115, and a console 2116 at the top of the elongated support structure 2114. The elongated support structure 2114 may include one or more carriages, such as a carriage 2117 (alternatively “arm support”) for supporting the deployment of one or more robotic arms 2112 (three shown in FIG. 2). The carriage 2117 may include individually configurable arm mounts that rotate along a perpendicular axis to adjust the base of the robotic arms 2112 for better positioning relative to the patient. The carriage 2117 also includes a carriage interface 2119 that allows the carriage 2117 to vertically translate along the elongated support structure 2114.

The carriage interface 2119 is connected to the elongated support structure 2114 through slots, such as slot 2120, that are positioned on opposite sides of the elongated support structure 2114 to guide the vertical translation of the carriage 2117. The slot 2120 contains a vertical translation interface to position and hold the carriage at various vertical heights relative to the cart base 2115. Vertical translation of the carriage 2117 allows the cart 2110 to adjust the reach of the robotic arms 2112 to meet a variety of table heights, patient sizes, and physician preferences. Similarly, the individually configurable arm mounts on the carriage 2117 allow the robotic arm base 2121 of robotic arms 2112 to be angled in a variety of configurations.

The elongated support structure 2114 may include internal mechanisms, such as gears and motors, that are designed to use a vertically aligned lead screw to translate the carriage 2117 in a mechanized fashion in response to control signals generated in response to user inputs, e.g., inputs from the console 2116.

The robotic arms 2112 may generally include robotic arm bases 2121 and tool drivers 2122, separated by a series of linkages 2123 that are connected by a series of joints 2124, each joint including an independent actuator, each actuator including an independently controllable motor. Each independently controllable joint represents an independent degree of freedom available to the robotic arm. Each of the arms 2112 have seven joints, and thus provide seven degrees of freedom. A multitude of joints result in a multitude of degrees of freedom, allowing for “redundant” degrees of freedom. Redundant degrees of freedom allow the robotic arms 2112 to position their respective tool drivers 2122 at a specific position, orientation, and trajectory in space using different linkage positions and joint angles. This allows for the system to position and direct a robotic surgical tool from a desired point in space while allowing the clinician to move the arm joints into a clinically advantageous position away from the patient to create greater access while avoiding arm collisions.

The cart base 2115 balances the weight of the elongated support structure 2114, carriage 2117, and arms 2112 over the floor. Accordingly, the cart base 2115 houses heavier components, such as electronics, motors, power supply, as well as components that either enable movement and/or immobilize the cart. For example, the cart base 2115 includes rollable wheel-shaped casters 2125 that allow for the cart 2110 to easily move around the room prior to a procedure. After reaching the appropriate position, the casters 2125 may be immobilized using wheel locks to hold the cart 2110 in place during the procedure.

Positioned at a vertical end of elongated support structure 2114, the console 2116 allows for both a user interface for receiving user input and a display screen (or a dual-purpose device such as, for example, a touchscreen 2126) to provide the clinician with both pre-operative and intra-operative data. Potential pre-operative data on the touchscreen 2126 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 2116 may be positioned and tilted to allow a clinician to access the console from the side of the elongated support structure 2114 opposite carriage 2117. From this position, the clinician may view the console 2116, robotic arms 2112, and patient while operating the console 2116 from behind the cart 2110. As shown, the console 2116 also includes a handle 2127 to assist with maneuvering and stabilizing cart 2110.

The distal end of the system's robotic arms include the tool driver 2122 (alternatively referred to as “instrument drive mechanism” or “instrument device manipulator” (IDM)) that incorporate electro-mechanical means for actuating the robotic tool. A removable or detachable robotic tool can be releasably mounted to the tool driver 2122. The robotic tool can be devoid of any electro-mechanical components, such as motors. This dichotomy may be driven by the need to sterilize robotic surgical tools used in medical procedures, and the inability to adequately sterilize expensive capital equipment due to their intricate mechanical assemblies and sensitive electronics. Accordingly, the robotic surgical tools may be designed to be detached, removed, and interchanged from the instrument driver (and thus the system) for individual sterilization or disposal by the clinician or the clinician's staff. In contrast, the instrument drivers need not be changed or sterilized, and may be draped for protection.

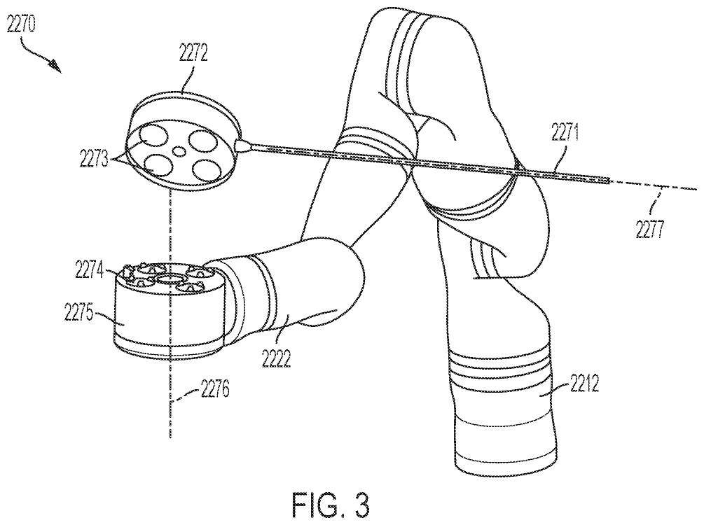

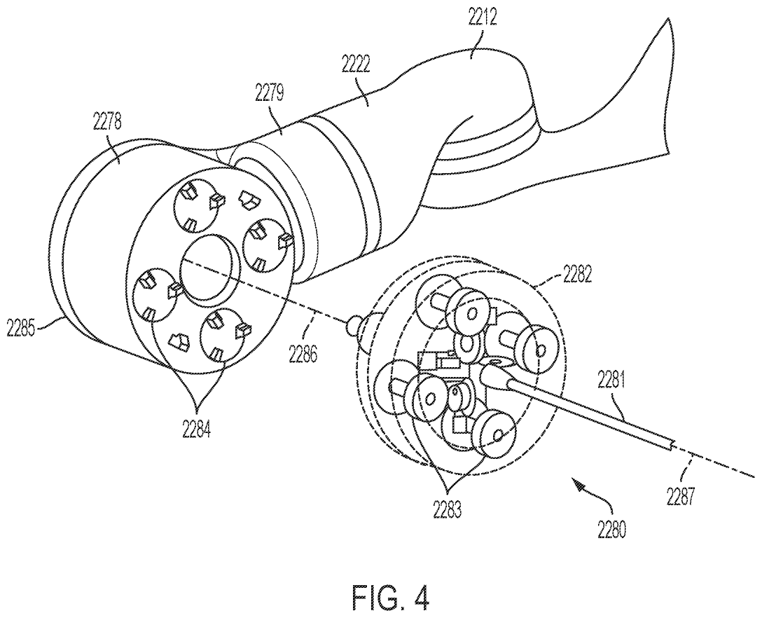

FIGS. 3 and 4 illustrate an example tool driver paired with a robotic surgical tool. The tool drivers are positioned at the distal end 2222 of a robotic arm 2212, which can be similar in many aspects to the robotic arms 2112. Positioned at the distal end 2222 of the robotic arm 2212, the tool drivers comprises one or more drive units arranged with parallel axes to provide controlled torque to a robotic surgical tool via drive shafts. Each drive unit includes an individual drive shaft for interacting with the instrument, a gear head for converting the motor shaft rotation to a desired torque, a motor for generating the drive torque, an encoder to measure the speed of the motor shaft and provide feedback to the control circuitry, and control circuitry for receiving control signals and actuating the drive unit. Each drive unit being independently controlled and motorized, the tool driver may provide multiple (four as shown in FIGS. 3 and 4) independent drive outputs to the robotic surgical tool. In operation, the control circuitry can receive a control signal, transmit a motor signal to the motor, compare the resulting motor speed as measured by the encoder with the desired speed, and modulate the motor signal to generate the desired torque, for example.

For procedures that require a sterile environment, the robotic system may incorporate a drive interface, such as a sterile adapter connected to a sterile drape, that sits between the tool driver and the robotic surgical tool. The chief purpose of the sterile adapter is to transfer angular motion from the drive shafts of the tool driver to the drive inputs of the instrument while maintaining physical separation, and thus sterility, between the drive shafts and drive inputs. Accordingly, an example sterile adapter may comprise of a series of rotational inputs and outputs intended to be mated with the drive shafts of the tool driver and drive inputs on the instrument. Connected to the sterile adapter, the sterile drape, comprised of a thin, flexible material such as transparent or translucent plastic, is designed to cover the capital equipment, such as the tool driver, the robotic arm, and the cart (in a cart-based system) or table (in a table-based system). Use of the drape would allow the capital equipment to be positioned proximate to the patient while still being located in an area not requiring sterilization (i.e., non-sterile field). On the other side of the sterile drape, the robotic surgical tool may interface with the patient in an area requiring sterilization (i.e., sterile field).

Robotic surgical platforms like the robotic surgical system 2100 are further described in U.S. Patent Application Publication No. 2021/0059777, titled ARTICULATING INCLUDING ANTAGONISTIC CONTROLS FOR ARTICULATION AND CALIBRATION, published Mar. 4, 2021. U.S. Patent Application Publication No. 2021/0059777, titled ARTICULATING INCLUDING ANTAGONISTIC CONTROLS FOR ARTICULATION AND CALIBRATION, published Mar. 4, 2021 is incorporated by reference herein in its entirety.

FIG. 3 depicts a robotic surgical tool 2270 with a paired tool driver 2275. The tool driver 2275 can be coupled to a distal end 2222 of the robotic arm 2212. Like other surgical tools designed for use with a robotic system, the robotic surgical tool 2270 includes an elongated shaft 2271 (or elongate body) and a housing (or base) 2272. The housing 2272, can also be referred to as an “instrument handle” due to its intended design for manual interaction by the clinician when attaching or coupling the surgical tool 2270 to the tool driver 2275 on the robotic arm 2212. The housing 2272 includes rotatable drive inputs 2273, e.g., receptacles, pulleys or spools, that are designed to be mated with drive outputs 2274 that extend through a drive interface on tool driver 2275 at the distal end 2222 of the robotic arm 2212. When physically connected, latched, and/or coupled, the mated drive inputs 2273 of housing 2272 may share axes of rotation with the drive outputs 2274 in the tool driver 2275 to allow the transfer of torque from drive outputs 2274 to drive inputs 2273. In some instances, the drive outputs 2274 may include splines that are designed to mate with receptacles on the drive inputs 2273. The drive outputs 2274 (and drive inputs 2273 when drivingly coupled thereto) are configured to rotate about axes parallel with a central axis 2276 defined through the tool driver 2275.

The elongated shaft 2271 is designed to be delivered through either an anatomical opening or lumen, e.g., as in endoscopy, or a minimally invasive incision, e.g., as in laparoscopy. The elongated shaft 2271 may be either flexible (e.g., having properties similar to an endoscope) or rigid (e.g., having properties similar to a laparoscope) or contain a customized combination of both flexible and rigid portions. In an unflexed configuration, the elongated shaft 2271 extends along a longitudinal axis 2277, which is transverse to the central axis 2276 of the tool driver 2275. When designed for laparoscopy, the distal end of a rigid elongated shaft may be connected to an end effector extending from a jointed wrist formed from a clevis with at least one degree of freedom and a surgical tool or robotic surgical tool, such as, for example, a grasper, scissors, a stapler, or other surgical device. The end effector can be actuated based on force from the tendons as the drive inputs 2273 rotate in response to torque received from the drive outputs 2274 of the tool driver 2275. Various highly articulatable robotic surgical tools are further described herein. When designed for endoscopy, the distal end of a flexible elongated shaft may include a steerable or controllable bending section that may be articulated and bent based on torque received from the drive outputs 2274 of the tool driver 2275.

Torque from the tool driver 2275 is transmitted down the elongated shaft 2271 using tendons along the shaft 2271. These individual tendons, such as pull wires, may be individually anchored to individual drive inputs 2273 within the housing 2272. From the housing 2272, the tendons are directed down one or more pull lumens along the elongated shaft 2271 and anchored at the distal portion of the elongated shaft 2271 or in the wrist at the distal portion of the elongated shaft 2271. During a surgical procedure, such as a laparoscopic, endoscopic or hybrid procedure, these tendons may be coupled to a distally mounted end effector, such as a grasper or scissors, for example. Under such an arrangement, torque exerted on drive inputs 2273 would transfer tension to the tendon, thereby causing the end effector to actuate in some way. In some instances, during a surgical procedure, the tendon may cause a joint to rotate about an axis, thereby causing the end effector to move in one direction or another. Alternatively, the tendon may be connected to one or more jaws of a grasper at distal end of the elongated shaft 2271, where tension from the tendon cause the grasper to close.

In endoscopy, the tendons may be coupled to a bending or articulating section positioned along the elongated shaft 2271 (e.g., at the distal end) via adhesive, a control ring, or other mechanical fixation. When fixedly attached to the distal end of a bending section, torque exerted on drive inputs 2273 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 2271 to allow for controlled articulation in the desired bending or articulable sections.

In endoscopy, the elongated shaft 2271 houses a number of components to assist with the robotic procedure. The shaft may include a working channel for deploying surgical tools (or robotic surgical tools), irrigation, and/or aspiration to the operative region at the distal end of the shaft 2271. The shaft 2271 may also accommodate wires and/or optical fibers to transfer signals to/from an optical assembly at the distal tip, which may include an optical camera. The shaft 2271 may also accommodate optical fibers to carry light from proximally-located light sources, such as light emitting diodes, to the distal end of the shaft. In various instances, an RF electrode can extend through the elongated shaft 2271 and can be configured to deliver RF energy to a distal end effector of the robotic surgical tool 2270.

At the distal end of the robotic surgical tool 2270, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

Referring still to FIG. 3, the drive shaft axes, and thus the drive input axes, are parallel to the central axis 2276 of the tool driver 2275 and orthogonal to the longitudinal axis 2277 of the elongated shaft. This arrangement, however, can complicate roll capabilities for the elongated shaft 2271 in certain instances. Rolling the elongated shaft 2271 along its longitudinal axis 2277 while keeping the drive inputs 2273 static may result in undesirable tangling of the tendons as they extend off the drive inputs 2273 and enter pull lumens within the elongated shaft 2271. The resulting entanglement of such tendons may disrupt any control algorithms intended to predict movement of the flexible elongated shaft during an endoscopic procedure.

FIG. 4 illustrates another tool driver 2285 and a paired robotic surgical tool 2280 where the axes of the drive units are parallel to an axis defined by an elongated shaft 2281 of the surgical tool 2280. As shown, a circular tool driver 2285 comprises four drive units with their drive outputs 2284 aligned in parallel at the end of the robotic arm 2212. The drive units, and their respective drive outputs 2284, are housed in a rotational assembly 2278 of the tool driver 2285 that is driven by one of the drive units within the rotational assembly 2278. In response to torque provided by the rotational drive unit, the rotational assembly 2278 rotates along a circular bearing that connects the rotational assembly 2278 to a non-rotational portion 2279 of the tool driver 2285. Power and controls signals may be communicated from the non-rotational portion 2279 of the tool driver 2285 to the rotational assembly 2278 through electrical contacts, which can be maintained through rotation by a brushed slip ring connection. In other aspects of the present disclosure, the rotational assembly 2278 may be responsive to a separate drive unit that is integrated into the non-rotational portion 2279, and thus not in parallel to the other drive units. The rotational assembly 2278 allows the tool driver 2285 to rotate the drive units, and their respective drive outputs 2284, as a single unit around a tool driver axis 2286.

Similar to the robotic surgical tool 2270, the robotic surgical tool 2280 includes an elongated shaft portion 2281 and a housing 2282 (shown as transparent in FIG. 4 for illustrative purposes) including a plurality of drive inputs 2283 (such as receptacles, pulleys, and spools) that are configured to receive the drive outputs 2284 in the tool driver 2285. Shaft 2281 extends from the center of the housing 2282 along a longitudinal axis 2287 substantially parallel to the axes of the drive inputs 2283, rather than orthogonal thereto as in the arrangement shown in FIG. 3.

When coupled to the rotational assembly 2278 of the tool driver 2285, the robotic surgical tool 2280, comprising the housing 2282 and shaft 2281, rotates in combination with the rotational assembly 2278 about a central axis 2286 defined through the tool driver 2285. Since the shaft 2281 is positioned at the center of the housing 2282, the shaft 2281 is coaxial with tool driver's central axis 2286 when attached. Thus, rotation of the rotational assembly 2278 causes the shaft 2281 to rotate about its own longitudinal axis 2287. Moreover, as the rotational assembly 2278 rotates with the shaft 2281, any tendons connected to the drive inputs 2283 in the housing 2282 are not tangled during rotation. Accordingly, the parallelism of the axes of the drive outputs 2284, drive inputs 2283, and shaft 2281 allows for the shaft rotation without tangling any control tendons.

In other instances, the tool drives may include a different configuration of actuated drives. For example, U.S. Patent Application Publication No. 2019/0201111, titled DRIVE ARRANGEMENTS FOR ROBOTIC-ASSISTED SURGICAL PLATFORMS, which published on Jul. 4, 2019, describes tool carriages having various drive arrangements. U.S. Pat. No. 9,072,535, titled SURGICAL STAPLING INSTRUMENTS WITH ROTATABLE STAPLE DEPLOYMENT ARRANGEMENTS, which issued Jul. 7, 2015, also describes tool carriages having various drive arrangements. U.S. Pat. No. 9,072,535, titled SURGICAL STAPLING INSTRUMENTS WITH ROTATABLE STAPLE DEPLOYMENT ARRANGEMENTS, which issued Jul. 7, 2015, and U.S. Patent Application Publication No. 2019/0201111, titled DRIVE ARRANGEMENTS FOR ROBOTIC-ASSISTED SURGICAL PLATFORMS, which published on Jul. 4, 2019, are incorporated by reference herein in their respective entireties. Alternative drive arrangements are further described herein.

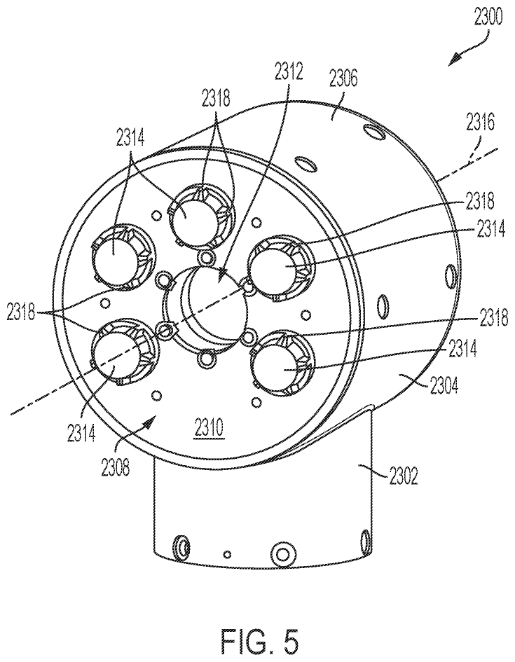

FIG. 5 depicts a perspective view of another tool driver 2300, which is also referred to herein as an IDM. The tool driver 2300 is similar in many aspects to the tool drivers 2285; however, the tool driver 2300 includes five rotary outputs. Various aspects of the tool driver 2300 are further described in U.S. Pat. No. 10,470,830, titled SYSTEM AND METHODS FOR INSTRUMENT BASED INSERTION ARCHITECTURES, issued Nov. 12, 2019, for example.

The tool driver 2300 can be used with the robotic surgical system 2100 and with the robotic arms 2212, for example. The tool driver 2300 is configured to attach a surgical tool to a robotic arm in a manner that allows the surgical tool to be continuously rotated, or “rolled”, about a longitudinal axis of the surgical tool. The tool driver 2300 includes a base 2302 and a surgical tool holder assembly 2304 coupled to the base 2302. The surgical tool holder assembly 2304 serves as a tool holder for holding a robotic surgical tool.

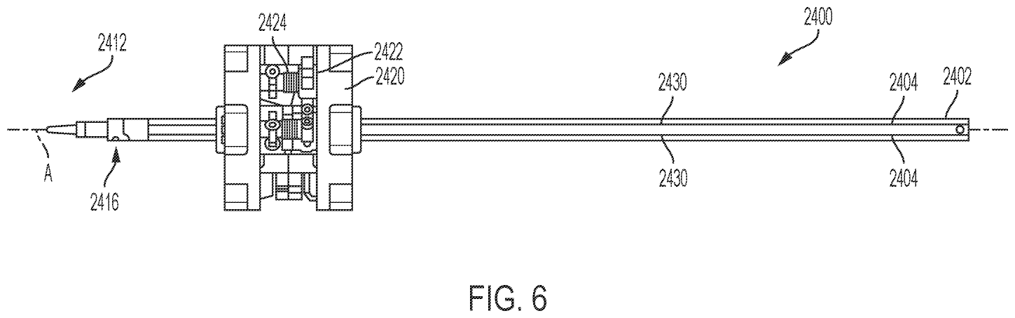

The surgical tool holder assembly 2304 further includes an outer housing 2306, a surgical tool holder 2308, an attachment interface 2310, a passage 2312, and a plurality of torque couplers 2314 that have splines 2318. The passage 2312 comprises a through-bore that extends from one face of the tool driver 2300 to an opposing face of the tool driver 2300 along a central axis 2316, which is collinear with a longitudinal axis of the surgical tool coupled thereto. The tool driver 2300 can be used with a variety of surgical tools, which may include a handle, or housing, and an elongated body, or shaft, and which may be for a laparoscope, an endoscope, or other types of surgical tools, such as electrosurgical tools including monopolar RF scissors, for example. An exemplary surgical tool 2400 is shown in FIG. 6, for example.

The base 2302 removably or fixedly mounts the tool driver 2300 to a robotic surgical arm of a robotic surgical system. In FIG. 5, the base 2302 is fixedly attached to the outer housing 2306 of the surgical tool holder assembly 2304. In alternative instances, the base 2302 is structured to include a platform, which is adapted to rotatably receive the surgical tool holder 2308 on the face opposite from the attachment interface 2310. The platform may include a passage aligned with the passage 2312 to receive the elongated body of the surgical tool and, in some instances, an additional elongated body of a second surgical tool mounted coaxially with the first surgical tool. One or more motors can be housed in the base 2302. For example, the surgical tool holder 2308 can include multiple motors, which are configured to drive, i.e. rotate output drives, also referred to herein as torque drivers and torque couplers, 2314 with a torque and rotary velocity, which can be controlled by the controller, for example.

The surgical tool holder assembly 2304 is configured to secure a surgical tool to the tool driver 2300 and rotate the surgical tool relative to the base 2302. Mechanical and electrical connections are provided from the surgical arm to the base 2302 and then to the surgical tool holder assembly 2304 to rotate the surgical tool holder 2308 relative to the outer housing 2306 and to manipulate and/or deliver power and/or signals from the surgical arm to the surgical tool holder 2308 and ultimately to the surgical tool. Signals may include signals for pneumatic pressure, electrical power, electrical signals, and/or optical signals.

The attachment interface 2310 is a face of the surgical tool holder 2308 that attaches to the surgical tool. The attachment interface 2310 includes a first portion of an attachment mechanism that reciprocally mates with a second portion of the attachment mechanism located on the surgical tool. The attachment interface 2310 is further described in U.S. Pat. No. 10,470,830, titled SYSTEM AND METHODS FOR INSTRUMENT BASED INSERTION ARCHITECTURES, issued Nov. 12, 2019, for example.

Various tools can attach to the tool driver 2300, including tools used for laparoscopic, endoscopic and endoluminal surgery. Tools can include tool-based insertion architectures that reduce the reliance on robotic arms for insertion. In other words, insertion of a surgical tool (e.g., towards a surgical site) can be facilitated by the design and architecture of the surgical tool. For example, in some instances, wherein a tool comprises an elongated shaft and a handle, the architecture of the tool enables the elongated shaft to translate longitudinally relative to the handle along an axis of insertion. Various advantages of tool-based insertion architectures are further described in U.S. Pat. No. 10,470,830, titled SYSTEM AND METHODS FOR INSTRUMENT BASED INSERTION ARCHITECTURES, issued Nov. 12, 2019, which is incorporated by reference herein its entirety.

A surgical tool 2400 having a tool-based insertion architecture is shown in FIG. 6. Various aspects of the surgical tool 2400 are further described in U.S. Pat. No. 10,470,830, titled SYSTEM AND METHODS FOR INSTRUMENT BASED INSERTION ARCHITECTURES, issued Nov. 12, 2019, for example.

The surgical tool 2400 enables a translation of the surgical tool 2400 (e.g., translation of its shaft 2402 and end effector 2412 relative to a tool driver and/or distal end of the robotic arm) along an insertion axis. In such instances, the surgical tool 2400 can be moved along the insertion axis without reliance—or with less reliance—on movement of a robotic arm. The surgical tool 2400 includes an elongated shaft 2402, an end effector 2412 connected to the shaft 2402, and a handle 2420, which may also be referred to as an instrument housing or base, coupled to the shaft 2402. The elongated shaft 2402 comprises a tubular member and includes one or more channels or grooves 2404 along its outer surface. The grooves 2404 are configured to receive one or more wires or cables 2430 therethrough. The cables 2430 run along an outer surface of the elongated shaft 2402. In other aspects of the present disclosure, certain cables 2430 can run through the shaft 2402 and may not be exposed. Manipulation of the cables 2430 (e.g., via the tool driver 2300) results in actuation of the end effector 2412, for example.

The end effector 2412 can include laparoscopic, endoscopic, or endoluminal components, for example, and can be designed to provide an effect to a surgical site. For example, the end effector 2412 can comprise a wrist, grasper, tines, forceps, scissors, clamp, knife, and/or fasteners. Exemplary surgical end effectors are further described herein. The cables 2430 that extend along the grooves on the outer surface of the shaft 2402 can actuate the end effector 2412. The cables 2430 extend from a proximal portion of the shaft 2402, through the handle 2420, and toward a distal portion of the shaft 2402, where they actuate the end effector 2412.

The instrument housing 2420 includes an attachment interface 2422 having one or more mechanical inputs 2424, e.g., receptacles, pulleys or spools, that are designed to be reciprocally mated with one or more torque couplers 2314 (FIG. 5) on the attachment interface 2310 of the tool driver 2300. The attachment interface 2422 is capable of attaching to the tool driver 2300 via a front-mount, back-mount and/or top mount. When physically connected, latched, and/or coupled together, the mated mechanical inputs 2424 of the instrument handle 2420 may share axes of rotation with the torque couplers 2314 of the tool driver 2300, thereby allowing the transfer of torque from the motors in the tool driver 2300 to the instrument handle 2420. In some instances, the torque couplers 2314 may comprise splines that are designed to mate with receptacles on the mechanical inputs. Cables 2430 that actuate the end effector 2412 engage the receptacles, pulleys, or spools of the handle 2420, such that the transfer of torque from the tool driver 2300 to the instrument handle 2420 results in actuation of the end effector 2412.

The surgical tool 2400 can include a first actuation mechanism that controls actuation of the end effector 2412. The surgical tool 2400 can also include a second actuation mechanism that enables the shaft 2402 to translate relative to the handle 2420 along an axis of insertion A. One or more additional actuation mechanism can effect articulation of the end effector 2412 relative to the shaft 2402. For example, the surgical tool 2400 can include an articulation joint 2416, which can allow articulation of the end effector 2412 relative to the shaft 2402 about one or more axes (e.g. pitch and yaw).

In various instances, an actuation mechanism can include one or more pulleys mounted on a rotary axis to change relative cable length and, in other instances, mounting a pulley on a lever, gear or track-based system to adjust its location. Additionally or alternatively, ball spline rotary shafts that travel down a length of a tool can also be used to transmit forces in a mechanically-remote way. Various actuation mechanisms are further described in U.S. Pat. No. 10,470,830, titled SYSTEM AND METHODS FOR INSTRUMENT BASED INSERTION ARCHITECTURES, issued Nov. 12, 2019, for example.

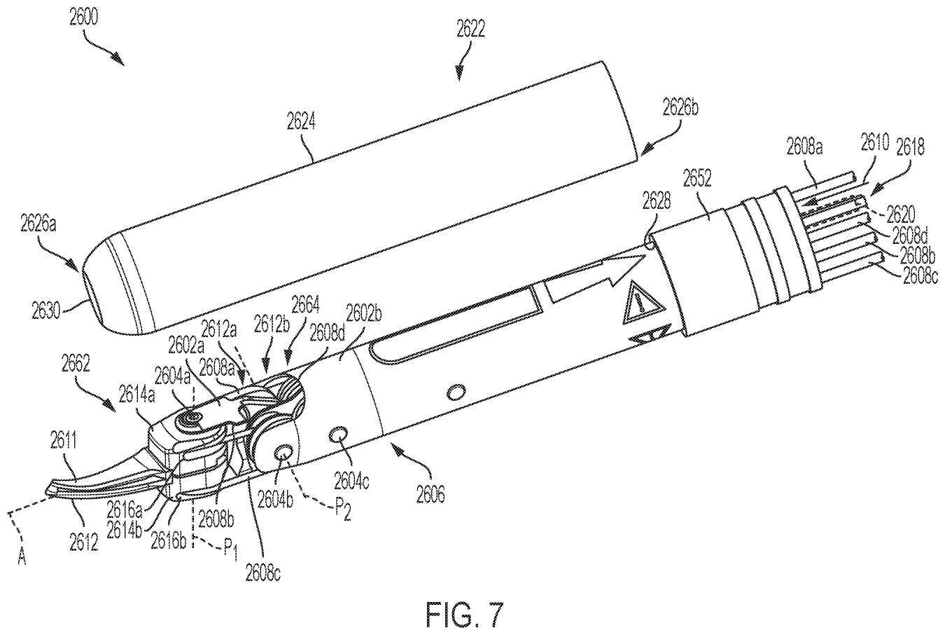

FIG. 7 depicts a distal portion of a surgical tool 2600. The surgical tool 2600 is similar in many aspects to the surgical tool 2400. For example, the surgical tool 2600 can have a tool-based insertion architecture in certain instances. In various instances, the surgical tool 2600 can be coupled to the robotic arm 2112 and/or the robotic arm 2212 via a tool driver, such as the tool driver 2275, the tool driver 2285, and/or the tool driver 2300, for example. The surgical tool 2600 includes an end effector 2662, a shaft 2652, and a wrist/articulation joint 2664 at which the surgical tool 2600 is configured to articulate. The end effector 2662 is shown in an unarticulated position in FIG. 7; however, the end effector 2662 is configured to be articulated at the wrist 2664 relative to the shaft. As used herein, the term “operatively couple” refers to a direct or indirect coupling engagement.

To operatively couple the end effector 2662 to the shaft 2652, the wrist 2664 includes a distal clevis 2602a and a proximal clevis 2602b. The end effector 2662, including the jaws 2611, 2612 thereof, is rotatably mounted to the distal clevis 2602a at a first axle 2604a, the distal clevis 2602a is rotatably mounted to the proximal clevis 2602b at a second axle 2604b, and the proximal clevis 2602b is coupled to a distal end 2606 of a portion of the shaft 2652.

The wrist 2664 provides a first pivot axis P₁ that extends through the first axle 2604a and a second pivot axis P₂ that extends through the second axle 2604b. The first pivot axis P₁ is substantially perpendicular (orthogonal) to the longitudinal axis A of the end effector 2662, and the second pivot axis P₂ is substantially perpendicular (orthogonal) to both the longitudinal axis A and the first pivot axis P₁. Movement about the first pivot axis P₁ provides “yaw” articulation of the end effector 2662, and movement about the second pivot axis P₂ provides “pitch” articulation of the end effector 2662. In the embodiment depicted in FIG. 7, the jaws 2611, 2612 are mounted at the first pivot axis P₁, thereby allowing the jaws 2611, 2612 to pivot relative to each other to open and close the end effector 2662 or alternatively pivot in tandem to articulate the orientation of the end effector 2662.

A plurality of drive cables, shown as drive cables 2608a, 2608b, 2608c, and 2608d, extend longitudinally within a lumen 2610 and pass through the wrist 2664 to be operatively coupled to the end effector 2662. While four drive cables 2608a-d are depicted in FIG. 6, more or less than four drive cables 2608a-d may be included without departing from the scope of the disclosure.

The drive cables 2608a-d form part of a cable driven motion system, and may be referred to and otherwise characterized as cables, bands, lines, cords, wires, ropes, strings, twisted strings, elongate members, etc. The drive cables 2608a-d can be made from a variety of materials including, but not limited to, metal (e.g., tungsten, stainless steel, etc.) or a polymer, for example. The lumen 2610 can be a single lumen, as shown in FIG. 7 or can comprise a plurality of independent lumens that each receive one or more of the drive cables 2608a-d.

The drive cables 2608a-d extend proximally from the end effector 2662 to the housing and are operatively coupled to various actuation mechanisms or devices housed (contained) therein to facilitate longitudinal movement (translation) of the drive cables 2608a-d within the lumen 2610. Selective actuation of all or a portion of the drive cables 2608a-d causes the end effector 2662 (e.g., one or both of the jaws 2611, 2612) to articulate (pivot) relative to the shaft 2652. More specifically, selective actuation causes a corresponding drive cable 2608a-d to translate longitudinally within the lumen 2610 and thereby cause pivoting movement of the end effector 2662. One or more drive cables 2608a-d, for example, may translate longitudinally to cause the end effector 2662 to articulate (e. g., both of the jaws 2611, 2612 angled in a same direction), to cause the end effector 2662 to open (e.g., one or both of the jaws 2611, 2612 move away from the other), or to cause the end effector 2662 to close (e.g., one or both of the jaws 2611, 2612 move toward the other).

Moving the drive cables 2608a-d can be accomplished in a variety of ways, such as by triggering an associated actuator or mechanism operatively coupled to or housed within the housing (e.g. the housing 2420 (FIG. 6)). Moving a given drive cable 2608a-d constitutes applying tension (i.e., pull force) to the given drive cable 2608a-d in a proximal direction relative to the end effector 2662, which causes the given drive cable 2608a-d to translate and thereby cause the end effector 2662 to move (articulate) relative to the shaft 2652.

The wrist 2664 includes a first plurality of pulleys 2616a and a second plurality of pulleys 2616b, each configured to interact with and redirect the drive cables 2608a-d for engagement with the end effector 2662. The first plurality of pulleys 2616a is mounted to the proximal clevis 2602b at the second axle 2604b and the second plurality of pulleys 2616b is also mounted to the proximal clevis 2602b but at a third axle 2604c located proximal to the second axle 2604b. The first and second pluralities of pulleys 2616a and 2616b cooperatively redirect the drive cables 2608a-d through an “S” shaped pathway before the drive cables 2608a-d are operatively coupled to the end effector 2662.

In at least one aspect, one pair of drive cables 2608a-d is operatively coupled to each jaw 2611, 2612 and configured to “antagonistically” operate the corresponding jaw 2611, 2612. In the embodiment depicted in FIG. 7, for example, the first and second drive cables 2608a and 2808b are coupled with a connector at the first jaw 2611, and the third and fourth drive cables 2608c and 2608d are coupled with a connector at the second jaw 2612. Consequently, actuation of the first drive cable 2608a pivots the first jaw 2611 about the first pivot axis P₁, toward the open position, and actuation of the second drive cable 2608b pivots the first jaw 2611 about the first pivot axis P₁ in the opposite direction and toward the closed position. Similarly, actuation of the third drive cable 2608c pivots the second jaw 2612 about the first pivot axis P₁ toward the open position, while actuation of the fourth drive cable 2608d pivots the second jaw 2612 about the first pivot axis P₁ in the opposite direction and toward the closed position.

Accordingly, the drive cables 2608a-d may be characterized or otherwise referred to as “antagonistic” cables that cooperatively (yet antagonistically) operate to cause relative or tandem movement of the first and second jaws 2611, 2612. When the first drive cable 2608a is actuated (moved), the second drive cable 2608b naturally follows as coupled to the first drive cable 2608a, and when the third drive cable 2608c is actuated, the fourth drive cable 2608d naturally follows as coupled to the third drive cable 2608c, and vice versa. Antagonistic cable mechanisms are further described in U.S. Patent Application Publication No. 2021/0059777, titled ARTICULATING INCLUDING ANTAGONISTIC CONTROLS FOR ARTICULATION AND CALIBRATION, published Mar. 4, 2021, which is incorporated by reference here in its entirety.

The end effector 2662 further includes a first jaw holder 2614a and a second jaw holder 2614b laterally offset from the first jaw holder 2614a. The first jaw holder 2614a is mounted to the first axle 2604a and configured to receive and seat the first jaw 2611 such that movement (rotation) of the first jaw holder 2614a about the first pivot axis P₁ correspondingly moves (rotates) the first jaw 2611. The first jaw holder 2614a may also provide and otherwise define a first pulley 2616a configured to receive and seat one or more drive cables, such as the first and second drive cables 2608a,b to effect such movement (rotation).

The second jaw holder 2614b is similarly mounted to the first axle 2604a and is configured to receive and seat the second jaw 2612 such that movement (rotation) of the second jaw holder 2614b about the first pivot axis P₁ correspondingly moves (rotates) the second jaw 2612. The second jaw holder 2614b may also provide and otherwise define a second pulley 2616b configured to receive and seat one or more drive cables, such as the third and fourth drive cables 2608c,d, to effect such movement (rotation).

The term “jaw holder”, as used herein, is intended to apply to a variety of types of end effectors having opposing jaws or blades that are movable relative to one another. In the depicted embodiment, the jaws 2611, 2612 comprise opposing scissor blades of a surgical scissors end effector. Accordingly, the jaw holders 2614a,b may alternately be referred to as “blade holders”. In other aspects of the present disclosure, however, the jaws 2611, 2612 may alternatively comprise opposing jaws used in a grasper end effector, or the like, and the term “jaw holder” similarly applies, without departing from the scope of the disclosure. Moreover, the term “holder” in “jaw holder” may be replaced with “mount”, “drive member”, or “actuation member”. For example,

The surgical tool 2600 may also include an electrical conductor 2618 that supplies electrical energy to the end effector 2662, thereby converting the surgical tool 2600 into an “electrosurgical instrument”. Similar to the drive cables 2608a-d, the electrical conductor 2618 may extend longitudinally within the lumen 2610. In some instances, the electrical conductor 2618 and a power cable may comprise the same structure. In other instances, however, the electrical conductor 2618 may be electrically coupled to a power cable, such as at the handle 270. In yet other instances, the electrical conductor 2618 may extend to the handle 270 where it is electrically coupled to an internal power source, such as a power cable, batteries, or fuel cells.

In some aspects of the present disclosure, the electrical conductor 2618 may comprise a wire. In other aspects, however, the electrical conductor 2618 may comprise a rigid or semi-rigid shaft, rod, or strip (ribbon) made of a conductive material. In some aspects, the electrical conductor 2618 may be partially covered with an insulative covering 2620 (shown in dashed lines) made of a non-conductive material. The insulative covering 2620, for example, may comprise a plastic applied to the electrical conductor 2618 via heat shrinking, but could alternatively be any other non-conductive material.

In operation, the end effector 2662 may be configured for monopolar or bipolar operation, without departing from the scope of the disclosure. Electrical energy is transmitted by the electrical conductor 2618 to the end effector 2662, which acts as an active (or source) electrode. In at least one aspect of the present disclosure, the electrical energy conducted through the electrical conductor 2618 may comprise radio frequency (“RF”) energy exhibiting a frequency between about 100 kHz and 5 MHz. The RF energy causes ultrasonic agitation or friction, in effect resistive heating, thereby increasing the temperature of target tissue. Accordingly, electrical energy supplied to the end effector 2662 is converted to heat and transferred to adjacent tissue to cut, cauterize, and/or coagulate the tissue (dependent upon the localized heating of the tissue), and thus may be particularly useful for sealing blood vessels or diffusing bleeding.

The surgical tool 2600 may further include a protective sleeve 2622 configured to insulate various live (energized) portions of the end effector 2662 (including the wrist 2664), and thereby protect the patient from stray electrical discharge during operation. As illustrated, the sleeve 2622 may comprise an elongate and generally cylindrical body 2624 having a first or distal end 2626a and a second or proximal end 2626b opposite the distal end 2626a. The body 2624 may be sized to extend over portions of the end effector 2662, the wrist 2664, and the shaft 2652. When the sleeve 2622 is properly positioned for use, the jaw members 2611, 2612 protrude out an aperture 2630 defined in the distal end 2626a of the body 2624 and the proximal end 2626b engages or comes into close contact with a radial shoulder 2628 defined on the shaft 2652. In this position, the sleeve 2622 is configured to ensure electrical current is only conducted to tissue as intended at the exposed jaw members 2611, 2612.

The sleeve 2622 may be assembled onto the tool 2600 within the sterile field before surgery and removed before cleaning the tool 2600. The sleeve 2622 must be properly installed to mitigate electrical discharge in unintended pathways, and the responsibility for proper installation is often left to the various clinicians on hand in an operating room (e.g. nurses).

Moreover, the sleeve 2622 is generally made of a flexible material and can be installed via an interference fit between the inner radial surface of the sleeve 2622 and the outer radial surfaces of the end effector 2662, the wrist 2664, and/or the shaft 2652. The flexibility of the sleeve 2622 allows the wrist 2664 to articulate during use. As the wrist 2664 articulates, however, the sleeve 2622 may have a tendency to creep axially, which results in the proximal end 2626b separating from the radial shoulder 2628 and increasing the likelihood of electrical discharge in unintended pathways. In certain instances, the robotic surgical tool 2600 can include a positive indicator that the sleeve 2622 has moved from its properly assembled position. Consequently, a clinician (e. g., a surgeon) may be alerted that electrical discharge in unintended pathways to the patient tissue may potentially ensue, thus prompting action to properly resituate the sleeve 2622 if warranted.

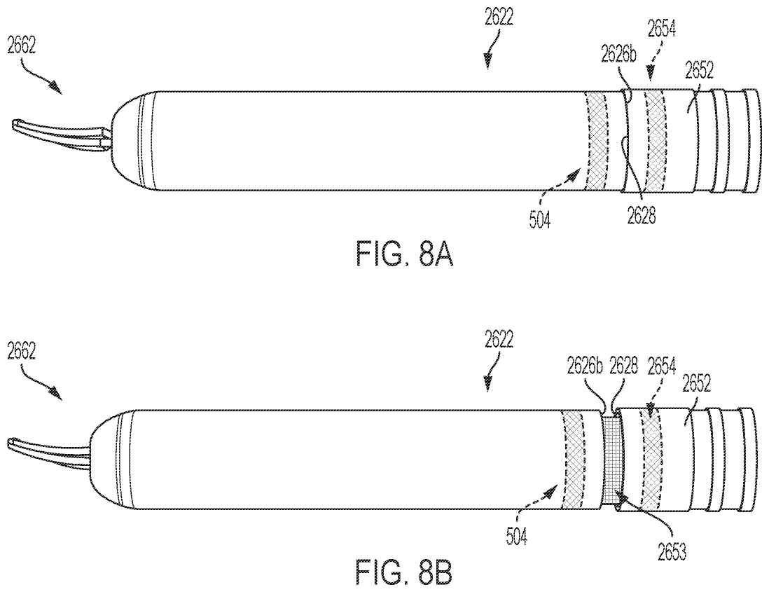

FIG. 8A depicts the sleeve 2622 in a first or “assembled” position, where the sleeve 2622 is properly positioned on the end effector 2662 for operation, and FIG. 8B depicts the sleeve 2622 in a second or “migrated” position, where the sleeve 2622 has moved (migrated) axially from the assembled position and relative to at least one of the end effector 2662 and the shaft 2652. In certain instances, an indicator 2653 may become exposed and thereby provide a positive indicator that the sleeve 2622 has transitioned from the assembled position. In some instances, the surgical tool 2600 may further include an over-assembled indicator 2654 (shown in dashed lines). The over-assembled indicator 2654 may be similar to the indicator 2653 except that it is used to help the clinician detect when the sleeve 2622 has been advanced (or migrated) proximally past the assembled position, which may comprise another type of migrated position but in the proximal direction. Various indicators are further described in U.S. Patent Application Publication No. 2019/0314107, published Oct. 17, 2019, titled PROTECTION MEASURES FOR ROBOTIC ELECTROSURGICAL INSTRUMENTS, which is incorporated by reference herein in its entirety.

Electrosurgical instruments are further described in U.S. Patent Application Publication No. 2019/0105099, titled ELECTRICAL ISOLATION OF ELECTROSURGICAL INSTRUMENT, published Apr. 11, 2019; U.S. Patent Application Publication No. 2019/0292291, published Sep. 19, 2019, titled SUPPLYING ELECTRICAL ENERGY TO ELECTROSURGICAL INSTRUMENTS; and U.S. Patent Application Publication No. 2019/0314107, published Oct. 17, 2019, titled PROTECTION MEASURES FOR ROBOTIC ELECTROSURGICAL INSTRUMENTS, which are each incorporated by reference herein in their respective entireties.

Even with a protective outer sheath appropriately positioned as further described herein, current may leak from an electrosurgical tool and arc to patient tissue in certain instances. For example, monopolar surgical devices often operate with a potential or working voltage in the range of 1,500-5,000 volts and at a frequency of about 400 kHz in certain instances. Moreover, unlike bipolar surgical devices having a return path therethrough, a monopolar surgical device may not have such a return path within the surgical device. In such instances, when a monopolar surgical device is energized but the end effector is not in contact with tissue, RF energy can capacitively couple to adjacent tissue through the protective outer sheath.

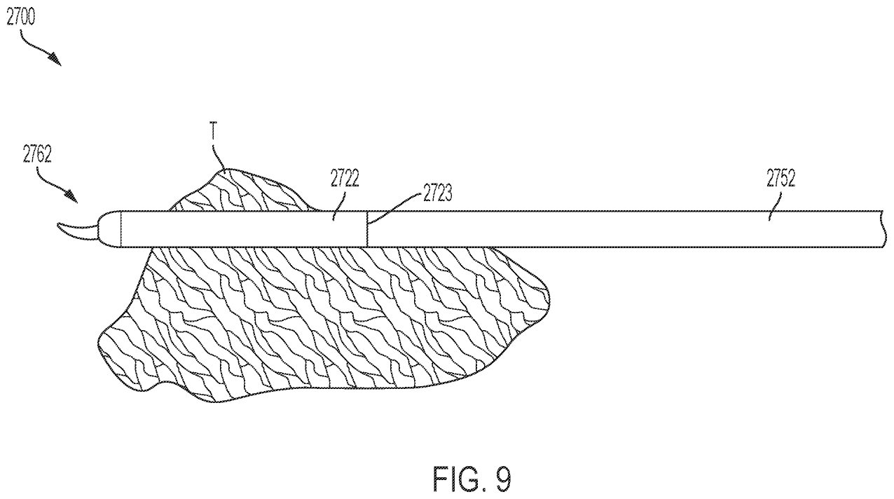

Referring now to FIG. 9, a distal portion of a surgical tool 2700 is shown. The surgical tool 2700 is similar in many aspects to the surgical tool 2600. The surgical tool 2700 can be coupled to the robotic arm 2112 and/or the robotic arm 2212 via a tool driver, such as the tool driver 2275, the tool driver 2285, and/or the tool driver 2300, for example, and/or can have a tool-based insertion architecture in certain instances. The surgical tool 2700 includes an end effector 2762, a shaft 2752, and a protective outer sheath 2722 extending around a portion of the end effector 2762 and the shaft 2752. In various instances, the surgical tool 2700 can be a pair of monopolar scissors and the end effector 2762 can include energizable scissor blades. A wrist/articulation joint can be positioned at a distal end of the shaft 2752 and/or proximal end of the end effector 2762 and surrounded by the protective outer sheath 2722, which is configured to flex as the end effector 2762 articulates relative to the shaft 2752.

In FIG. 9, the end effector 2762 is not in contact with tissue T; however, a portion of the protective other sheath 2722 and the shaft 2752 are positioned in abutting contact with tissue. When the end effector 2762 is energized with a monopolar waveform, current can arc through the protective outer sheath 2722 to the tissue T in certain instances. For example, current within the protective outer sheath 2722 can capacitatively couple to tissue adjacent to a proximal end 2723 of the protective outer sheath 2722. Inadvertent current leakage despite the appropriate placement of an electrically-insulative protective outer sheath 2722 can unintentionally energize/heat tissue and may damage tissue in certain instances. Current may be more prone to arcing and/or leakage in surgical devices having a large proportion of metal components, such as electrosurgical scissors, for example. Additionally or alternatively, high RF frequencies and voltage potentials of monopolar devices, for example, can increase the likelihood of current arc/leakage in certain instances.

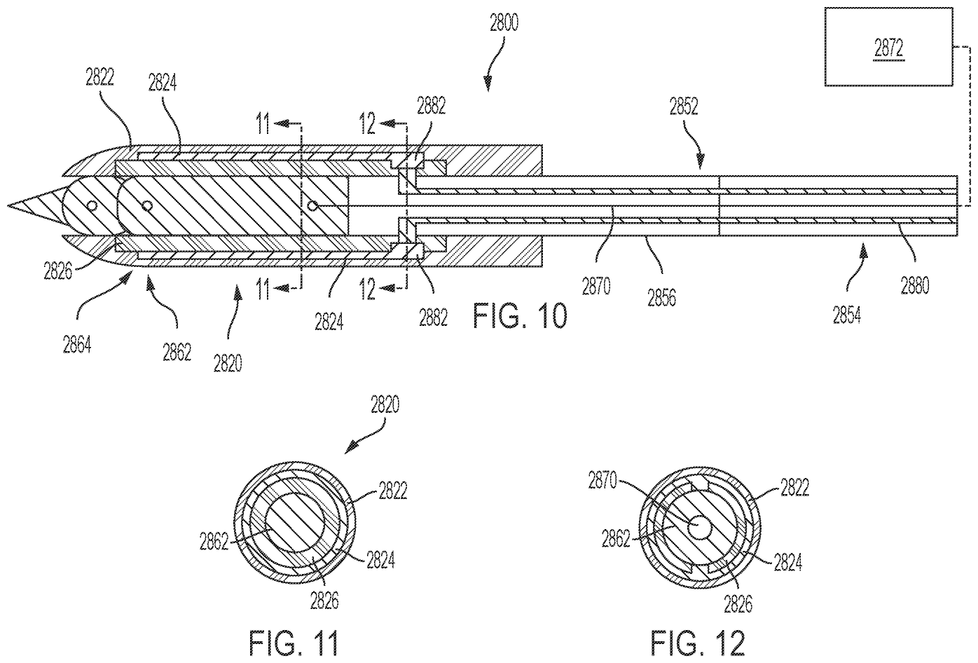

Referring now to FIGS. 10-12, a distal portion of a surgical tool 2800 is shown. The surgical tool 2800 is similar in many aspects to the surgical tool 2600. For example, in various instances, the surgical tool 2800 can be a pair of monopolar scissors having energizable scissor blades at the distal end thereof. Additionally or alternatively, the surgical tool 2800 can be coupled to the robotic arm 2112 and/or the robotic arm 2212 via a tool driver, such as the tool driver 2275, the tool driver 2285, and/or the tool driver 2300, for example, and/or can have a tool-based insertion architecture in certain instances.

In other instances, the surgical tool 2800 can include graspers, tines, forceps, scissors, clamp, knife, and/or fasteners. In still other aspects of the present disclosure, the surgical tool 2800 can be a monopolar hook, monopolar spatula, pencil, or other electrosurgical tool without opposing jaw holders.

The surgical tool 2800 includes an end effector 2862, a shaft assembly 2852, and a protective sheath assembly 2820 extending around a portion of the end effector 2862 and a portion of the shaft assembly 2852. The shaft assembly 2852 includes an electrically-conductive proximal shaft segment 2854 and an electrically-insulative distal shaft segment 2856. The electrically-insulative distal shaft segment 2856 can be adjacent to the end effector 2862, which is selectively energized by an electrosurgical generator 2872 during use. A wrist/articulation joint 2864 is positioned at the proximal end of the end effector 2862 and surrounded by the protective sheath assembly 2820, which is configured to flex as the end effector 2862 articulates relative to the shaft 2852. In other aspects of the present disclosure, the wrist/articulation joint 2864 is positioned at the distal end of the distal electrically-insulative shaft segment 2856. The articulation joint 2864 can include a series of devises and pivot pins, as further described herein, which can facilitate articulation of the end effector 2862 about two or more different axes in various aspects of the present disclosure.

An RF conductor 2870 extends through the shaft assembly 2852 distally to the end effector 2862. The RF conductor 2870 is configured to selectively deliver RF current to the end effector 2862 from the electrosurgical generator 2872. The RF conductor 2870 can deliver voltage in the range of 1,500-5,000 volts and at a frequency of about 400 kHz during certain monopolar activations, for example.

A guard electrode 2880 also extends distally through the shaft assembly 2852 to an electrical contact region 2882 at which the guard electrode 2880 terminates within the electrically-insulative distal shaft segment 2856. The guard electrode 2880 acts as a shield around the RF conductor 2870. For example, the guard electrode 2880 can define a tubular conduit through which the RF conductor 2870 extends. In other instances, the guard electrode 2880 can define a non-insulative wire or tape wrapping and/or extending along an electrically-insulative material within the shaft assembly 2852. In certain instances, the guard electrode 2880 comprises a wound shielded cable. For example, the guard electrode can be similar to a coaxial cable in various aspects of the present disclosure. An electrically-insulative layer is also positioned between the RF conductor 2870 and the guard electrode 2880. For example, the RF conductor 2870 can define a multi-layer construction having an insulative outer layer. In various instances, the RF conductor 2870 is a stranded wire, for example, having an insulative material, e.g. a polymeric material, heat-shrunk therearound. In certain instances, the RF conductor can include a traditional shielded cable construction. In various instances, the RF conductor 2870 can include hypodermic tubing (hypotube) heat-shrunk to a copper wire, for example.

As further described herein, the surgical tool 2800 includes a protective sheath assembly 2820, which is configured to electrically insulate a proximal portion of the end effector 2862. For example, the wrist/articulation joint 2864 can be electrically insulated; however, the distal blades can be exposed to grasp and/or transect tissue. T

The protective sheath assembly 2820 includes multiple layers. For example, the protective sheath assembly 2820 includes an electrically-insulative outer layer 2822, an electrically-insulative inner layer 2826, and an electrically-conductive layer 2824 intermediate the electrically-insulative outer layer 2822 and electrically-insulative inner layer 2826. The electrically-conductive intermediate layer 2824 is configured to shunt electrical current along an inner lumen of the surgical tool 2800 to the guard electrode 2880 and ultimately back to the electrosurgical generator 2872 to prevent arcing of current along portions of the surgical tool 2800 proximal to the tissue-treatment region, e.g. proximal to the distal blades of the end effector 2862. The multi-layer protective sheath 2820 can be installed on the surgical tool as further described herein with respect to the protective sheath 2622 and the surgical tool 2600 (FIGS. 7-8B), for example.

The electrically-insulative outer layer 2822 forms the distal-most and proximal-most ends of the protective sheath assembly 2820 such that the electrically-conductive layer 2824 is encased within and surrounded by the electrically-insulative outer layer 2822. Consequently, the electrically-insulative outer layer 2822c shields the electrically-conductive layer 2824 from tissue contact. In certain instances, the electrically-insulative outer layer 2822 comprises heat-shrunk tubing around the electrically-conductive layer 2824. In certain instances, the electrically-insulative outer layer 2822 comprises thermoplastic polyurethane (TPU) and/or silicone molded or extruded over the electrically-conductive layer 2824.

Similarly, the electrically-insulative inner layer 2826 extends distally beyond the electrically-conductive layer 2824 and proximally to the contact region 2882 along the electrically-insulative distal shaft segment 2856. In such instances, the electrically-insulative inner layer 2826 shields the electrically-conductive layer 2824 from contact with the end effector 2862. In certain instances, a portion of the electrically-insulative inner layer 2826 and/or the electrically-insulative outer layer 2822 can hook around the electrically-conductive layer 2824 to enclose and shield a distal end of the electrically-conductive layer 2824. In other instances, the electrically-insulative inner layer 2826 can be incorporated into the end effector 2862 and can include an insulative coating thereon, for example.

The electrically-conductive layer 2824 extends proximally and terminates within the electrically-insulative outer layer 2822 at the contact region 2882. The electrically-conductive layer 2824 is electrically coupled to the guard electrode 2880 at the contact region 2882 and provides a return path for stray current back through the shaft assembly 2852 to the electrosurgical generator 2872. In various instances, the return path via the electrically-conductive layer 2824, contact region 2882, and guard electrode 2880 can be electrically isolated from the distally-flowing current path defined by the RF conductor 2870 and the end effector 2862. Moreover, the return path via the electrically-conductive layer 2824, contact region 2882, and guard electrode 2880 can be electrically isolated from an exterior surface of the surgical tool 2800 (e.g. encased within the shaft 2852) and, thus, isolated from patient tissue, in various instances. Unlike the return path in a bipolar device, the return path does not terminate at a tissue treatment region in the end effector 2862, but rather terminates at an insulated distal end within the electrically-insulative outer layer 2822. The primary return path for a monopolar instrument remains the return pad affixed to the patient. The secondary return path described above, which includes the contact region 2882, is fully insulated from the patient and from the primary return path. The secondary return path serves as a return path only for any leakage current.

The contact region 2882 comprises an opening in the electrically-insulative distal shaft segment 2856 though which current is configured to flow to the guard electrode 2880. In various instances, the contact region 2882 can include a radial compression fit. A proximal end and/or proximal rivet of the electrically-conductive layer 2824 can be press-fit into an opening in the electrically-insulative distal shaft segment 2856. In other instances, a distal end and/or distal rivet of the guard electrode 2880 can be press-fit into a radial opening in the electrically-insulative distal shaft segment 2856.

The multi-layer protective sheath assembly 2820 and guard electrode 2880 can be used in connection with monopolar scissors as further described herein. Alternative electrosurgical tools—both monopolar and bipolar—are also contemplated including various highly articulatable surgical tools. For example, the multi-layer protective sheath assembly 2820 and guard electrode 2880 can be incorporated into hooks, spatulas, pencils, and combination monopolar/suction irrigation devices, for example. Additionally, in various instances, the multi-layer protective sheath assembly 2820 and guard electrode 2880 can be incorporated into a robotic surgical tool and/or a handheld electrosurgical instrument. Such capacitive coupling mitigation techniques can be utilized whenever an energizable distal end effector is supported by an elongate shaft, for example.

### EXAMPLES

Various aspects of the subject matter described herein are set out in the following numbered examples.

Example 1—A monopolar surgical tool, comprising a shaft assembly comprising an electrically-conductive proximal portion and an electrically-insulative distal portion comprising a distal end. The monopolar surgical tool further comprises an end effector coupled to the distal end, an RF conductor extending distally through the shaft assembly to the end effector, and a guard electrode extending distally through the shaft assembly to an electrical contact region along the electrically-insulative distal portion, wherein the guard electrode surrounds the RF conductor along a length thereof. The monopolar surgical tool further comprises a multi-layer sheath assembly. The multi-layer sheath assembly comprises an electrically-insulative inner layer surrounding a portion of the end effector and extending proximally along a portion of the shaft assembly, an electrically-insulative outer layer, and an electrically-conductive layer positioned between the electrically-insulative inner layer and the electrically-insulative outer layer. The electrically-conductive layer extends proximally to the electrical contact region. The electrically-conductive layer provides a return path for leaked current to the electrical contact region and along the guard electrode within the shaft assembly.

Example 2—The monopolar surgical tool of Example 1, wherein the end effector comprises an articulation joint, wherein the multi-layer sheath assembly extends around the articulation joint, and wherein the electrical contact region is proximal to the articulation joint.

Example 3—The monopolar surgical tool of Examples 1 or 2, wherein the electrical contact region comprises an opening in the electrically-insulative distal portion of the shaft assembly.

Example 4—The monopolar surgical tool of Examples 1, 2, or 3, wherein the RF conductor is operably coupled to an electrosurgical generator, and wherein the guard electrode is configured to extend the return path for leaked current to the electrosurgical generator.

Example 5—The monopolar surgical tool of Examples 1, 2, 3, or 4, wherein the end effector comprises a first jaw and a second jaw, and wherein at least one of the first jaw and the second jaw is configured to pivot to transect tissue therebetween.

Example 6—The monopolar surgical tool of Examples 1, 2, 3, 4, or 5, wherein the end effector comprises electrosurgical scissors.

Example 7—The monopolar surgical tool of Examples 1, 2, 3, 4, 5, or 6, wherein the guard electrode comprises a tubular conduit.

Example 8—The monopolar surgical tool of Examples 1, 2, 3, 4, 5, 6, or 7, wherein the electrically-insulative outer layer comprises heat-shrunk tubing around the electrically-conductive layer.

Example 9—The monopolar surgical tool of Examples 1, 2, 3, 4, 5, 6, 7, or 8, further comprising a housing coupled to the shaft assembly and configured to receive rotary inputs from a robotic system, wherein the shaft assembly extends through the housing.

Example 10—A monopolar surgical tool, comprising a shaft, an electrically-conductive end effector comprising an articulation joint and a distal end, wherein the distal end is configured to articulate relative to the shaft at the articulation joint, and an RF conductor extending distally through the shaft to the electrically-conductive end effector. The monopolar surgical tool further comprises a guard electrode extending distally through the shaft to an electrical contact region, wherein the guard electrode surrounds the RF conductor along a length thereof. The monopolar surgical tool further comprises a sheath assembly. The sheath assembly comprises an electrically-insulative outer sheath surrounding the articulation joint and extending proximally along a portion of the shaft, and an electrically-conductive inner sheath electrically isolated from the electrically-conductive end effector and encased by the electrically-insulative outer sheath. The electrically-conductive inner sheath extends proximally to the electrical contact region and is configured to provide a return path for leaked current from the electrically-conductive inner sheath along the guard electrode through the shaft.

Example 11—The monopolar surgical tool of Example 10, wherein the shaft comprises an electrically-conductive proximal portion, and an electrically-insulative distal portion extending distally to the electrically-conductive end effector.

Example 12—The monopolar surgical tool of Example 11, wherein the electrical contact region forms a portion of the return path for leaked current through the electrically-insulative distal portion, wherein a primary return path comprises a patient pad, and wherein the return path for leaked current is electrically-isolated from the primary return path.

Example 13—The monopolar surgical tool of Examples 11 or 12, wherein the electrical contact region comprises an opening in the electrically-insulative distal portion.

Example 14—The monopolar surgical tool of Examples 10, 11, 12, or 13, wherein the RF conductor is operably coupled to a generator, and wherein the guard electrode is operably configured to extend the return path for leaked current from the electrical contact region to the generator.

Example 15—The monopolar surgical tool of Examples 10, 11, 12, 13, or 14, wherein the electrically-conductive end effector comprises a first jaw and a second jaw, and wherein at least one of the first jaw and the second jaw is configured to pivot to transect tissue therebetween.

Example 16—The monopolar surgical tool of Examples 10, 11, 12, 13, 14, or 15, wherein the electrically-conductive end effector comprises electrosurgical scissors.

Example 17—The monopolar surgical tool of Examples 10, 11, 12, 13, 14, 15, or 16, wherein the guard electrode comprises a wound shielded cable.

Example 18—The monopolar surgical tool of Examples 10, 11, 12, 13, 14, 15, 16, or 17, wherein the electrically-insulative outer sheath comprises a molded material selected from a group consisting of silicone and thermoplastic polyurethane.

Example 19—The monopolar surgical tool of Examples 10, 11, 12, 13, 14, 15, 16, 17, or 18, further comprising a housing coupled to the shaft and configured to receive rotary inputs from a robotic system, wherein the shaft extends through the housing.

Example 20—A monopolar surgical tool for use with a robotic surgical system and a generator. The monopolar surgical tool comprises a shaft comprising an electrically-insulative distal portion, an electrically-conductive distal end effector extending distally from the shaft, and an electrical conductor extending through the shaft to the electrically-conductive distal end effector. The monopolar surgical tool further comprises a capacitive coupling mitigation system configured to provide a secondary return path to the generator. The capacitive coupling mitigation system comprises a conductive contact region defined through the electrically-insulative distal portion, and a conductive inner sheath extending proximally from the electrically-conductive distal end effector to the conductive contact region. The conductive inner sheath is electrically isolated from the electrically-conductive distal end effector. The capacitive coupling mitigation system further comprises a guard electrode extending proximally from the conductive contact region through the shaft to the generator.

Example 21—The monopolar surgical tool of Example 20, wherein the electrically-conductive distal end effector comprises a pencil.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December, 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material. In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A monopolar surgical tool, comprising:
a shaft assembly comprising an electrically-conductive proximal portion and an electrically-insulative distal portion comprising a distal end;
an end effector coupled to the distal end;
an RF conductor extending distally through the shaft assembly to the end effector;
a guard electrode extending distally through the shaft assembly to an electrical contact region along the electrically-insulative distal portion, wherein the guard electrode surrounds the RF conductor along a length thereof; and
a multi-layer sheath assembly, comprising:
an electrically-insulative inner layer surrounding a portion of the end effector and extending proximally along a portion of the shaft assembly;
an electrically-insulative outer layer; and
an electrically-conductive layer positioned between the electrically-insulative inner layer and the electrically-insulative outer layer, wherein the electrically-conductive layer extends proximally to the electrical contact region, and wherein the electrically-conductive layer provides a return path for leaked current to the electrical contact region and along the guard electrode within the shaft assembly.

2. The monopolar surgical tool of claim 1, wherein the end effector comprises an articulation joint, wherein the multi-layer sheath assembly extends around the articulation joint, and wherein the electrical contact region is proximal to the articulation joint.

3. The monopolar surgical tool of claim 1, wherein the electrical contact region comprises an opening in the electrically-insulative distal portion of the shaft assembly.

4. The monopolar surgical tool of claim 1, wherein the RF conductor is operably coupled to an electrosurgical generator, and wherein the guard electrode is configured to extend the return path for leaked current to the electrosurgical generator.

5. The monopolar surgical tool of claim 1, wherein the end effector comprises a first jaw and a second jaw, and wherein at least one of the first jaw and the second jaw is configured to pivot to transect tissue therebetween.

6. The monopolar surgical tool of claim 5, wherein the end effector comprises electrosurgical scissors.

7. The monopolar surgical tool of claim 1, wherein the guard electrode comprises a tubular conduit.

8. The monopolar surgical tool of claim 1, wherein the electrically-insulative outer layer comprises heat-shrunk tubing around the electrically-conductive layer.

9. The monopolar surgical tool of claim 1, further comprising a housing coupled to the shaft assembly and configured to receive rotary inputs from a robotic system, wherein the shaft assembly extends through the housing.

10. A monopolar surgical tool, comprising:
a shaft;
an electrically-conductive end effector comprising an articulation joint and a distal end, wherein the distal end is configured to articulate relative to the shaft at the articulation joint;
an RF conductor extending distally through the shaft to the electrically-conductive end effector;
a guard electrode extending distally through the shaft to an electrical contact region, wherein the guard electrode surrounds the RF conductor along a length thereof; and
a sheath assembly, comprising:
an electrically-insulative outer sheath surrounding the articulation joint and extending proximally along a portion of the shaft; and
an electrically-conductive inner sheath electrically isolated from the electrically-conductive end effector and encased by the electrically-insulative outer sheath, wherein the electrically-conductive inner sheath extends proximally to the electrical contact region and is configured to provide a return path for leaked current from the electrically-conductive inner sheath along the guard electrode through the shaft.

11. The monopolar surgical tool of claim 10, wherein the shaft comprises:
an electrically-conductive proximal portion; and
an electrically-insulative distal portion extending distally to the electrically-conductive end effector.

12. The monopolar surgical tool of claim 11, wherein the electrical contact region forms a portion of the return path for leaked current through the electrically-insulative distal portion, wherein a primary return path comprises a patient pad, and wherein the return path for leaked current is electrically-isolated from the primary return path.

13. The monopolar surgical tool of claim 12, wherein the electrical contact region comprises an opening in the electrically-insulative distal portion.

14. The monopolar surgical tool of claim 10, wherein the RF conductor is operably coupled to a generator, and wherein the guard electrode is operably configured to extend the return path for leaked current from the electrical contact region to the generator.

15. The monopolar surgical tool of claim 10, wherein the electrically-conductive end effector comprises a first jaw and a second jaw, and wherein at least one of the first jaw and the second jaw is configured to pivot to transect tissue therebetween.

16. The monopolar surgical tool of claim 15, wherein the electrically-conductive end effector comprises electrosurgical scissors.

17. The monopolar surgical tool of claim 10, wherein the guard electrode comprises a wound shielded cable.

18. The monopolar surgical tool of claim 10, wherein the electrically-insulative outer sheath comprises a molded material selected from a group consisting of silicone and thermoplastic polyurethane.

19. The monopolar surgical tool of claim 10, further comprising a housing coupled to the shaft and configured to receive rotary inputs from a robotic system, wherein the shaft extends through the housing.

20. A monopolar surgical tool for use with a robotic surgical system and a generator, comprising:
a shaft comprising an electrically-insulative distal portion;
an electrically-conductive distal end effector extending distally from the shaft;
an electrical conductor extending through the shaft to the electrically-conductive distal end effector; and
a capacitive coupling mitigation system configured to provide a secondary return path to the generator, wherein the capacitive coupling mitigation system comprises:
a conductive contact region defined through the electrically-insulative distal portion;
a conductive inner sheath extending proximally from the electrically-conductive distal end effector to the conductive contact region, wherein the conductive inner sheath is electrically isolated from the electrically-conductive distal end effector; and
a guard electrode extending proximally from the conductive contact region through the shaft to the generator.

21. The monopolar surgical tool of claim 20, wherein the electrically-conductive distal end effector comprises a pencil.
